(19) United States
(12) Patent Application Publication
(10) Pub. No.: US 2020/0054109 A1
(43) Pub. Date: Feb. 20, 2020

(54) SELF-TANNING APPLICATOR DEVICE AND METHOD OF MAKING THEREOF

(71) Applicant: Lori Braun, North Las Vegas, NV (US)

(72) Inventor: Lori Braun, North Las Vegas, NV (US)

(21) Appl. No.: 16/541,891

(22) Filed: Aug. 15, 2019

Related U.S. Application Data
(60) Provisional application No. 62/719,401, filed on Aug. 17, 2018.

(51) Int. Cl. A45D 34/04; B65D 75/58; A61K 8/60; A61Q 19/04

(52) U.S. Cl. CPC A45D 34/04; B65D 75/5805; A45D 2200/1036; A61Q 19/04; A45D 2200/1018; A61K 8/60

(57) ABSTRACT

An applicator device includes an applicator mitt. The applicator mitt includes a non-permeable layer configured to receive a user's hand when the non-permeable layer is in an open configuration. A fabric layer extends around the non-permeable layer and is impregnated with a liquid material. A packaging element surrounds the applicator mitt. The packaging element seals the non-permeable layer in a closed configuration. The packaging element is configured to be opened to provide the non-permeable layer in the open configuration. A method of applying a liquid material using the applicator device and a method of making the applicator device are also disclosed.

This application claims the benefit of U.S. Provisional Patent Application Ser. No. 62/719,401, filed Aug. 17, 2018, which is hereby incorporated by reference in its entirety.

### FIELD

The present technology relates to a self-tanning applicator device and methods of making and use thereof.

### BACKGROUND

The average person looks upon a tan as a sign of healthy attractiveness and exposure to the outdoors. However, in recent years, medical science has come to appreciate the dangers of exposure to the sun, particularly UVA exposure (320-400 nm of the solar spectrum band) and UVB radiation exposure (290-320 nm). There has been a sharp increase in the frequency of cancer occurring in exposed areas, such as basal cell carcinoma, squamous cell carcinoma and, most dangerous of all, malignant melanoma.

The dangers noted above have recently increased the public's interest in self-tanning compositions. In early versions, self-tanning compositions tended to result in an orange-like appearance which most people found unacceptable. However, even present self-tanning compositions can give the user an ‘orange’ or ‘fake’ appearance due to the relatively large amount of composition applied onto the hand and thereafter applied to the body part to be treated.

Present self-tanning compositions are sold as liquids, creams, and gels. Drops are applied to the user's hand, which then serves to apply the solution to the portion of the body, e.g. face, upper torso, etc. to be “tanned.” Streaking of the body portion due to uneven application typically occurs. The texture of the user's hands also factors into this streaking effect. Furthermore, this method generally results in an orange or brown pigment being left on the applicant's hands. The user's hands need to be thoroughly scrubbed to avoid later discoloration.

There is also a certain amount of waste in applying the composition to the hand and then onto the body, as well as maintaining containers or bottles of seldom used solution. Most current self-tanners have been found to leave stains on clothes, furniture, etc. if not given time to dry effectively. Treated body parts must be kept clear of such materials for 0.5 to 1.0 hour after application.

Tanning applicators have been developed to avoid having to transfer the self-tanning composition from the hand to the body directly. However, such applicators still require some contact between the user's hand and the self-tanning composition, which requires the user to thoroughly wash his or her hand after use to avoid discoloration.

The present technology is directed to overcoming these and other deficiencies in the art.

### SUMMARY

One aspect of the present technology relates to a an applicator device. The applicator device includes an applicator mitt. The applicator mitt includes a non-permeable layer configured to receive a user's hand when the non-permeable layer is in an open configuration. A fabric layer extends around the non-permeable layer and is impregnated with a liquid material. A packaging element surrounds the applicator mitt. The packaging element seals the non-permeable layer in a closed configuration. The packaging element is configured to be opened to provide the non-permeable layer in the open configuration.

Another aspect of the present technology relates to a method of applying a liquid material using the applicator device. The method includes opening the packaging element surrounding the applicator mitt. A hand is inserted into the non-permeable layer of the applicator mitt such that the hand does not contact the fabric layer. The applicator mitt is removed from the packaging element. The liquid material is applied to a surface by contacting the fabric layer to the surface.

A further aspect of the present technology relates to a method of making an applicator device. The method includes providing an applicator mitt. The applicator mitt includes a non-permeable layer configured to receive a user's hand when the non-permeable layer is in an open configuration and a fabric layer extending around the non-permeable layer. The applicator mitt is sealed. The sealed applicator mitt is inserted into a packaging element. The packaging element is filled with a liquid material such that the liquid material is in contact with the fabric layer of the sealed applicator mitt. The packaging element is sealed, wherein the packaging element and applicator mitt are configured to be opened to provide the non-permeable layer in the open configuration.

The present technology provides a number of advantages including providing a self-tanning applicator device, methods of applying a self-tanning using the self-tanning applicator device, and methods of making the self-tanning applicator device that allow for the application of a self-tanning material or composition without the need to contact the user's hand with the self-tanning composition. The self-tanning applicator device advantageously includes a packaging that contains the self-tanning composition in an outer fabric layer of an applicator mitt. The packaging precludes the composition from entering the interior surface of the applicator mitt. The user can open the packaging to remove the applicator mitt from the packaging and apply the self-tanning composition without the composition contacting the user's hand. The applicator device of the present technology provides an inexpensive, single-use self-tanning applicator that delivers a more efficient and clean application of a self-tanning composition.

### BRIEF DESCRIPTION OF THE DRAWINGS

FIG. 1 is a front partial phantom view of an exemplary self-tanning applicator device of the present technology with an applicator mitt located in a packaging.

FIG. 2 is a side cross-sectional view of the exemplary self-tanning applicator device shown in FIG. 1.

FIG. 3 is a top view of the exemplary self-tanning applicator device shown in FIG. 1 with the applicator mitt in an open configuration.

### DETAILED DESCRIPTION OF THE INVENTION

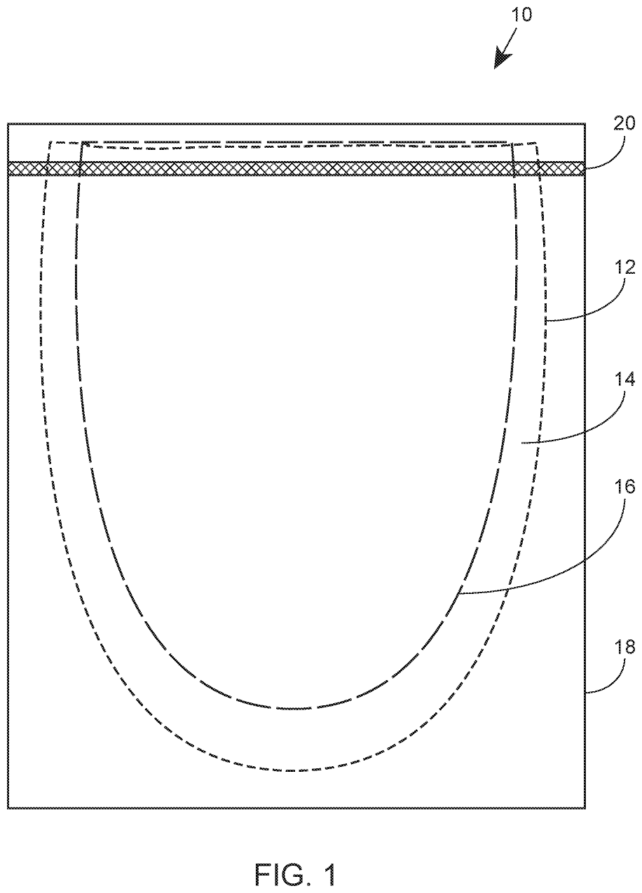

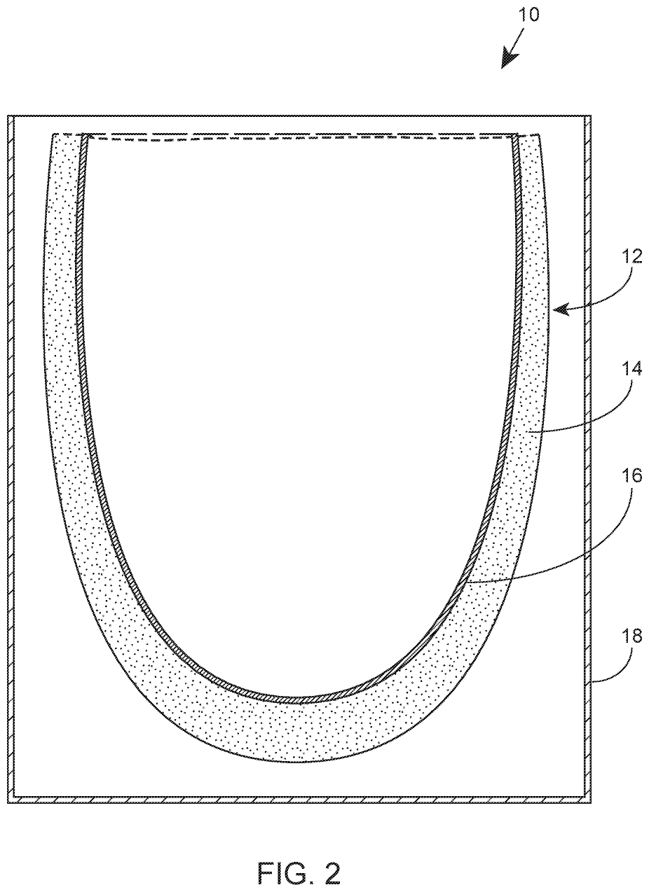

An exemplary self-tanning applicator device 10 of the present technology is illustrated in FIGS. 1-3. In this example, the self-tanning applicator device 10 includes an applicator mitt 12 including an outer fabric layer 14 and an inner non-permeable layer 16, and an outer packaging 18, although the self-tanning applicator device 10 may include other types and/or numbers of devices, elements, and/or components in other configurations. The self-tanning applicator device 10 provides a number of advantages including providing a self-tanning applicator that allows a user to apply the self-tanning composition using his or her hand without the hand contacting the self-tanning composition. Thus, the self-tanning composition is applied only to the desired skin surfaces.

In this example, the applicator mitt 12 is configured to receive a user's hand when in an open configuration as described in further detail below. The applicator mitt 12 may have any suitable size and shape to allow a user to insert his or hand into the applicator mitt 12 during use. Although the applicator mitt 12 is shown lacking a thumb portion, in other examples the applicator mitt 12 may be formed as a glove that includes a specific region for placement of the user's thumb. In this example, the applicator mitt 12 includes the outer fabric layer 14 and the inner non-permeable layer 16, although the applicator mitt 12 may include other types and/or numbers of layers arranged in other configurations.

The outer fabric layer 14 surrounds the outer surface of the applicator mitt 12. The outer fabric layer 14 is formed from synthetic or natural fibers, or mixtures thereof. In one example, the outer fabric layer 14 is a nonwoven material, such as spunlace. In one example, the outer fabric layer comprises at least 80% rayon and at least 20% polyethylene terephalate, although other combinations are contemplated.

The outer fabric layer 14 absorbs a self-tanning composition. In another example the outer fabric layer 14 is impregnated with the self-tanning composition. Although self-tanning composition is described, the outer fabric layer 14 could include other types of liquids. In one example, the outer fabric layer includes a self-tanning composition typically comprising dihydroxyacetone as the key active ingredient. In one example, the outer fabric layer 14 may be utilized with the materials described in U.S. Pat. No. 5,972,360, the disclosure of which is hereby incorporated herein by reference in its entirety. In some examples, the self-tanning composition may be clear, while in other examples the self-tanning composition or material may be tinted. The self-tanning composition incorporated in the outer fabric layer 14 can additionally contain skin conditioners, emollients, and the like. The outer fabric layer 14 may also include an appreciable amount of wood pulp fibers so as to provide exfoliation of dead skin during application of the tanning solution. Thus a cleansing effect as well as a tanning effect is obtained. Although the present technology is described with respect to a self-tanning composition, it is to be understood that the technology could be utilized with other liquid products, including other liquid beauty products, designed to be applied to user's skin.

The inner non-permeable layer 16 is located underneath the outer fabric layer 14 and is configured to receive the user's hand during use. In one example, the outer fabric layer 14 is heat bonded and sealed to the inner non-permeable layer 16. The inner non-permeable layer 16 may be formed of any suitable non-permeable material, such as leakproof foil, typically made of aluminum, silver polyester laminate, polyester laminate, or combinations thereof, although other materials such as non-permeable pliable plastics can be utilized. In one example, the inner non-permeable layer 16 includes various layers of material in combination, such as polyethylene terephalate, aluminum, and cast polypropolene film. In one example, the inner non-permeable layer 16 includes a polyethylene terephalate layer with a thickness of about 12 microns, an aluminum layer with a thickness of about 6 microns, and cast polypropolene film layer with a thickness of about 60 microns, although other combinations may be contemplated with other thicknesses. Inner non-permeable layer 16 substantially prevents the self-tanning composition incorporated in the outer fabric layer 14 from entering the inner portion of the applicator mitt 12 to prevent the self-tanning composition from contacting the user's hand during use. The inner non-permeable layer 16 is coupled to the outer fabric layer 14 using known manufacturing techniques, such as heat bonding. In one example, the outer fabric layer 14 is sealed around the edges of the inner non-permeable layer 16 such that the applicator mitt 12 must be cut to provide the user access to the inner non-permeable layer 16.

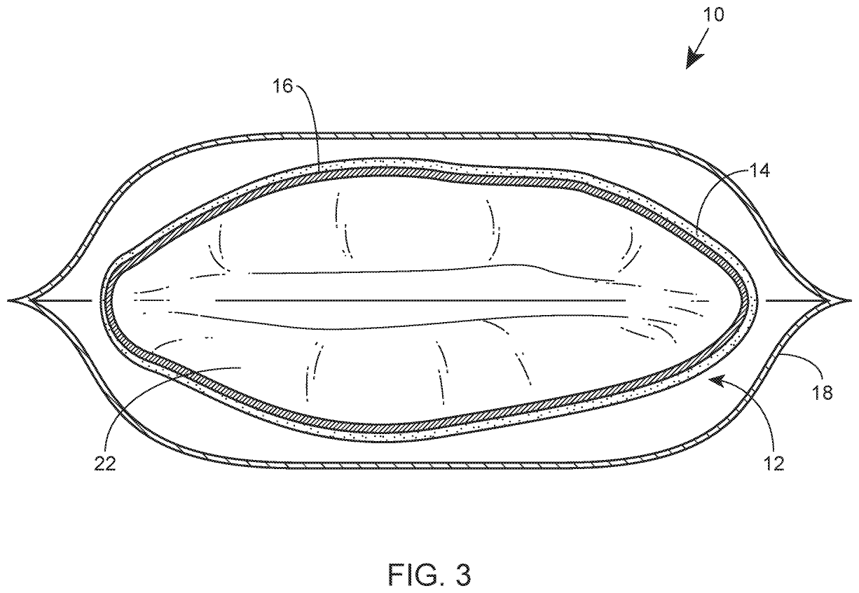

The outer packaging 18 is configured to house the applicator mitt 12. The outer packaging 18 is a formed of a leakproof foil, typically made of aluminum, silver polyester laminate, or polyester laminate with a foil lining, although the outer packaging 18 may be formed of other leak-proof materials. In this example, the packaging 18 is rectangular in shape, although the packaging 18 may have other shapes and sizes suitable to encapsulate the applicator mitt 12. The outer packaging 18 is ultrasonically sealed such that the self-tanning composition is sealed within the outer packaging 18. Outer packaging 18 includes a perforation or line 20 that may be cut to provide access to the applicator mitt 12. The perforation or line 20 also allows the sealed applicator mitt 12 to be cut to provide the inner non-permeable layer 16 in an open configuration for the user to insert a hand into the inner non-permeable layer 16 for use. Referring to FIG. 3, when the outer packaging 18 and the applicator mitt are cut along the perforation 20, an opening 22 is formed that allows for insertion of the user's hand into the inner non-permeable layer 16.

An exemplary operation of the self-tanning applicator device 10 will now be described with respect to FIGS. 1-3. First, the user opens the outer packaging 18 by cutting or tearing along the perforation or line 20. Cutting or tearing along perforation or line 20 opens the packaging and creates the opening 22 (as shown in FIG. 3) for insertion of the user's hand into inner non-permeable layer 16.

Next, the user inserts his or her hand into the non-permeable layer 16 of the applicator mitt 12. The design of the applicator mitt 12 allows the user to avoid contact with the outer fabric layer 14 that is impregnated with the self-tanning composition. The user then removes the applicator mitt 12 from the outer packaging 18.

The user then applies the self-tanning material or composition impregnated in the surface of the outer fabric layer 14 to a skin surface by contacting the outer fabric layer 14 to the desired skin surface. The user can wipe the outer fabric layer 14 over the skin surface, e.g., the user's face, neck or other body part, to have a tanned appearance. The user can then discard the applicator mitt 12. A natural glow will develop in one to three hours. For greater tanning, the user can utilize another self-tanning applicator device 10. One application every few days will preserve a tanned appearance.

A method of making the self-tanning applicator device 10 includes providing the applicator mitt 12 including the non-permeable layer 16 and the outer fabric layer 14 extending around the inner non-permeable layer 16. The applicator mitt 12 may be formed using known manufacturing techniques for coupling the outer fabric layer 14 to the non-permeable layer 16. In one example, the outer fabric layer 14 is heat bonded to the inner non-permeable layer 16. In one example, the applicator mitt 12 is formed using a spunlace nonwoven material. In other examples, the applicator mitt 12 may be formed with other types and/or numbers of additional layers. In this example, the applicator mitt 12, including the outer fabric layer 14 and the inner non-permeable layer 16 are ultrasonically sealed together around the edges of applicator mitt 12, such that the non-permeable layer 16 is sealed in a closed configuration.

Next, applicator mitt 12 is inserted into the outer packaging 18. The outer packaging 18 is then filled with a liquid, such as a self-tanning composition as described above. The outer packaging 18 is filled with the self-tanning composition such that the composition surrounds and is in contact with the outer fabric layer 14 of the sealed applicator mitt 12. The self-tanning composition does not enter the sealed applicator mitt 12 and instead only is absorbed on the surface of the outer fabric layer 14. In other examples, the outer fabric layer 14 is impregnated with the self-tanning composition as described in U.S. Pat. No. 5,972,360, the disclosure of which is hereby incorporated herein by reference in its entirety, although other impregnation techniques known in the art may be employed.

In this example, the applicator mitt 12 is then sealed in the outer packaging 18 with the self-tanning composition inside. In one example, the outer packaging is ultrasonically sealed to maintains the self-tanning composition within the outer packaging 18. The perforation or line 20 is provided on the outer packaging 18 to indicate where the outer packaging 18 can be opened. The perforation or line 20 also indicates where the user can cut to also cut the sealed applicator mitt 12 to provide the inner non-permeable layer 16 in the open configuration as described above and shown in FIG. 3.

Accordingly, the present technology provides a number of advantages including providing a self-tanning applicator device that allows for the application of a self-tanning material or composition without the need to apply or contact the user's hand with the self-tanning composition.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. An applicator device comprising:
an applicator mitt comprising:
a non-permeable layer configured to receive a user's hand when the non-permeable layer is in an open configuration; and
a fabric layer extending around the non-permeable layer and impregnated with a liquid material; and
a packaging element surrounding the applicator mitt, the packaging element sealing the non-permeable layer in a closed configuration, wherein the packaging element is configured to be opened to provide the non-permeable layer in the open configuration.

2. The device of claim 1, wherein the fabric layer is formed from synthetic or natural fibers.

3. The device of claim 2, wherein the fabric layer is formed of a nonwoven fabric material.

4. The device of claim 1, wherein the liquid material is a self-tanning material that comprises dihydroxyacetone.

5. The device of claim 4, wherein the liquid material comprises a coloring agent, a skin conditioner, an emollient, or an exfoliating material.

6. The device of claim 1, wherein the packaging element is ultrasonically sealed.

7. The device of claim 6, wherein the packaging element comprises a perforation that may be cut to provide the non-permeable layer in the open configuration.

8. A method of applying a liquid material using the device of claim 1, the method comprising:
opening the packaging element surrounding the applicator mitt;
inserting a hand into the non-permeable layer of the applicator mitt, wherein the hand does not contact the fabric layer;
removing the applicator mitt from the packaging element; and
applying the liquid material to a surface by contacting the fabric layer to the surface.

9. The method of claim 8, wherein the liquid material is a self-tanning material that comprises dihydroxyacetone.

10. The method of claim 9, wherein the liquid material comprises a coloring agent, a skin conditioner, an emollient, or an exfoliating material.

11. The method of claim 8, wherein the surface comprises a skin surface.

12. The method of claim 8, wherein the packaging element is ultrasonically sealed.

13. The method of claim 12, wherein the opening the packaging element comprises cutting the packaging element along a perforation to provide the non-permeable layer in the open configuration.

14. A method of making a self-tanning applicator device, the method comprising:
providing an applicator mitt comprising:
a non-permeable layer configured to receive a user's hand when the non-permeable layer is in an open configuration; and
a fabric layer extending around the non-permeable layer;
sealing the applicator mitt;
inserting the sealed applicator mitt into a packaging element;
filling the packaging element with a liquid material such that the liquid material is in contact with the fabric layer of the sealed applicator mitt; and
sealing the packaging element, wherein the packaging element and applicator mitt are configured to be opened to provide the non-permeable layer in the open configuration.

15. The method of claim 14, wherein the fabric layer is formed from synthetic or natural fibers.

16. The method of claim 15, wherein the fabric layer is formed of a nonwoven fabric material.

17. The method of claim 14, wherein the liquid material is a self-tanning material that comprises dihydroxyacetone.

18. The method of claim 17, wherein the liquid material comprises a coloring agent, a skin conditioner, an emollient, or an exfoliating material.

19. The method of claim 14, wherein the packaging element is ultrasonically sealed.

20. The method of claim 14 further comprising forming a perforation on the packaging element that may be cut to provide the non-permeable layer in the open configuration.
